(19) United States
(12) Patent Application Publication
(10) Pub. No.: US 2008/0305537 A1
(43) Pub. Date: Dec. 11, 2008

(54) Microchips and its Manufacturing Methods Thereof

(76) Inventors: Setsuya Sato, Osaka (JP); Yoshitaka Matsumoto, Kyoto-shi (JP); Toshio Teramoto, Tsuchiura-shi (JP); Kimitaka Morohoshi, Yokohama-shi (JP)

Correspondence Address: LAW OFFICES OF ALBERT WAI-KIT CHAN, PLLC, WORLD PLAZA, SUITE 604, 141-07 20TH AVENUE, WHITESTONE, NY, 11357, US

(21) Appl. No.: 11/944,059

(22) Filed: Nov. 21, 2007

Related U.S. Application Data
(63) Continuation-in-part of application No. PCT/JP2006/310150, filed on May 22, 2006.

(30) Foreign Application Priority Data
May 23, 2005 (JP) JP 2005-149653

(51) Int. Cl. C12M 1/14; C23C 14/54

(52) U.S. Cl. 435/287.2; 427/9

(57) ABSTRACT

A microchip is provided with a lower substrate configured as the lower portion of the microchip, an intermediate section formed on the top of the lower substrate, and an upper substrate formed on the top of the intermediate section, wherein the lower substrate, the intermediate section, and the upper substrate are made of light-transmissive and cured resin.

This application is a Continuation-In-Part Application of International Application No. PCT/JP2006/310150, filed on May 22, 2006, which claims priority of Japanese Patent Application No. 2005-149653, filed on May 23, 2005, the entire content and disclosure of the preceding applications are incorporated by reference into this application.

### FIELD OF THE INVENTION

The present invention relates to the microchips for handling micro objects such as micro chemical chips, iontophoretic chips, immune assay chips, or cellular chips, and manufacturing methods of those microchips.

### DESCRIPTION OF THE BACKGROUND ART

A various microchips with a cavity such as a flow channel therein have been proposed. They are, for example, μ-TAS (Micro Total Analytical System) or a substrate of several square-centimeters called “Lab. On a Chip”. Such substrates (microchips) are used for experimental purposes such as solution blending, reaction, separation, or detection.

Japanese publication No. 2004-210592 proposes a microchip comprising a couple of glass substrate. At least one of the glass substrate comprises a groove. By jointing these glass substrates to each other, a cavity is formed inside the microchip.

By making fluids or gases flow into the cavity formed in this way, operations such as analyses, chemical syntheses, or cell manipulations are conducted in the cavity.

A protrusion may be made in the cavity to perform the analyses requiring high precision or the complicated cell manipulations. The shape or arrangement of the protrusion is determined in accordance with desired analyses or cell manipulations.

As more precise analyses or more complicated cell manipulations are required, higher precision is required for determining the shape or the dimensions of this protrusion.

To form a protrusion in the cavity of the microchip disclosed in Japanese Patent Publication No. 2004-210592, etching processes is applied at the time of forming the groove. With this method, the shape of the protrusion is restricted to same specific shape. For example, it is difficult to form a cone-like protrusion with a vertical cross-section shaped as an upside-down triangle such that the tip thereof directly connects to the bottom of the groove.

In Japanese Patent Publication No. 2005-310757, it is described how to form a micro-three-dimensional structure using FIB (Focused Ion Beam) is formed.

The method described in Japanese Patent Publication No. 2005-310757 is summarized as follows: A microarea on the surface of the target object is scanned by FIB. The scan by FIB makes it possible to sputter the atoms on the surface of the target object, so that etching effect can be obtained. Consequently, minute cutting operation is enabled.

Furthermore, the spraying the phenanthrene gas (C₄H₁₀) onto the surface of the target object makes it possible to fix the components of the sprayed gas to a desired area on the surface of the target object. This allows to form a thin film fixed to the surface of the target object.

Thus, the preferable combination of the etching and the fixation of a thin film enables to obtain a minute three-dimensional structure in high precision.

The method disclosed in Japanese Patent Publication No. 2005-310757 is excellent in an aspect of obtaining a minute three-dimensional structure in a high precision. However, this method is not suitable for manufacturing microchips employed together with an optical microscope. That is to say, the thin film formed by spraying the phenantherene gas comprises diamond-like carbon, which is not light-transmissive.

Therefore, with the method disclosed in Japanese Patent Publication No. 2005-310757, it is not possible to effectively manufacture microchips for optical microscopes.

The present invention was developed in view of the above mentioned conventional case. The present invention allows desired microprocessing in a high precision, as well as aims at providing the microchips preferably usable for observation by optical microscopes or cell manipulations by optical devices, and manufacturing methods thereof.

### SUMMARY OF INVENTION

In one embodiment of the present invention, a microchip is provided with a lower substrate configured as the lower portion of the microchip, an intermediate section formed on the top of the lower substrate, and an upper substrate formed on the top of the intermediate section, wherein the lower substrate, the intermediate section, and the upper substrate are made of light-transmissive and cured resin, and are integrally formed.

In another embodiment of the present invention, the microchip is provided, wherein a cavity is formed in the intermediate section, a microstructure is protruded from the wall face of the cavity, and the microstructure is integrated with the wall face.

Yet in another embodiment of the present invention, the microchip is provided, wherein the lower substrate is formed in multiple rectangle-shaped blocks divided by grooves formed in a lattice pattern, wherein the intermediate section is formed in a thin plate having multiple apertures connecting with the grooves dividing the rectangle-shaped blocks, wherein the upper substrate has a honeycomb structure formed by connecting thin plate walls each other, wherein the internal space of the honeycomb structure connects with the apertures.

Yet in another embodiment of the present invention, the microchip is provided, wherein the lower substrate is formed in a plurality of aligned rectangle blocks divided by the grooves formed in a lattice pattern, wherein the intermediate section includes the multiple U-shaped thin walls on a plan view connected each other, wherein the upper substrate is formed to seal the top of the thin wall, wherein notches are formed on the top end of the thin wall.

Yet in another embodiment of the present invention, the microchip is provided, wherein the intermediate section in a trapezoidal cone shape comprises multiple hollow bars, wherein the upper substrate comprises multiple hollow bars that are extending upward from each of the multiple bars configured as the intermediate section, wherein the hollow bars configured as the intermediate section and the hollow bars configured as the upper substrate form a microcapillary, wherein an aperture that a cell is passable is formed on the periphery of the top end of the microcapillary, wherein at least one aperture is formed on the lower portion of the microcapillary than the cell passable aperture.

Yet in another embodiment of the present invention, a microchip is provided, wherein the lower substrate includes multiple apertures arrayed in matrix thereon and grooves arrayed in lattice pattern thereon, wherein the intermediate section includes multiple globular cavity apertures, wherein the upper substrate includes multiple apertures arrayed in matrix thereon and the grooves arrayed in lattice pattern thereon, wherein the apertures of the lower substrate, the cavity apertures of the intermediate section, and the cavity apertures of the upper substrate are communicated.

Yet in another embodiment of the present invention, the microchip is provided, wherein the laminated layer comprising the intermediate section with the cavity apertures and the upper substrate with the apertures and the grooves is further piled on the top of the upper substrate.

Yet in another embodiment of the present invention, a method of manufacturing microchip is provided comprising a formation process of the lower substrate with a certain thickness by curing the light-curing resin, a formation process of the intermediate section on the top face of the lower substrate in an integrated manner with the lower substrate, and a formation process of the upper substrate with a certain thickness on the top of the intermediate section in an integrated manner with the intermediate section by curing the light-curing resin, wherein during the forming process of the intermediate section, integrally laminating an additional cured resin layer on the other cured resin layer repeatedly by proceeding (a) dripping light-curing resin fluid onto the cured resin layer formed in the light-cured resin, (b) controlling an interval between the top of the cured resin layer and the bottom end of the fluid thickness control plate set above a stage where the lower substrate is set on, making a horizontal relative movement between the stage and the fluid thickness control plate, and attaching the resin fluid on the cured resin layer and the bottom end to control the even thickness of the fluid layer formed on the cured resin layer, and (c) irradiating a light to the fluid layer to cure the light-cured resin, wherein non-light-irradiation area and light-irradiation area are set in the step of the integral lamination during the formation process of the intermediate process to form three-dimensional space of the non-irradiation area, wherein a light is at least partially irradiated to three dimensional space to form a cavity and a microstructure protruded from a wall face of the cavity.

Yet in another embodiment of the present invention, the method of manufacturing microchip described is provided, wherein the non-light-irradiation area is set in the step of laminating layer in the formation process of the upper substrate to form three-dimensional space of the non-irradiation area, wherein the three-dimensional space formed in the upper substrate connects the three-dimensional space formed in the intermediate section with the outer surface of the upper substrate.

Yet in another embodiment of the present invention, the method of manufacturing microchip described is provided, wherein the non-light-irradiation area is set in the laminating layer in the formation process of the lower substrate to form three-dimensional space of the non-irradiation area, wherein the three-dimensional space formed in the lower substrate connects the three-dimensional space formed in the intermediate section with the outer surface of the lower substrate.

In one embodiment of the present invention, the lower substrate, intermediate section, and upper substrate are made of a light-transmissive and cured resin. Therefore, it is possible to add a minute shape on the lower substrate, intermediate section, and upper substrate. Furthermore, the microchip is preferably usable for cell observation or cell manipulation using an optical microscopes or optical devices.

In another embodiment of the present invention, it is possible to extremely improve the accuracy of the shape, arrangement, and array of the microstructure. Therefore, it is possible to perform chemical analyses and cell manipulations in a high precision.

Yet in another embodiment of the present invention, the microchip makes it possible to arrange cells regularly.

Yet in another embodiment of the present invention, the microchip makes it possible to capture the microparticles contained in the fluid, as well as minutely arrange the captured microparticles.

Yet in another embodiment of the present invention, the microchip makes it possible to separate a specific cell from multiple cells and take away the specific cell.

Yet in another embodiment of the present invention, the microchip makes it possible to arrange the cells regularly.

Yet in another embodiment of the present invention, the microchip makes it possible to regularly arrange the cells in the three-dimensional way.

Yet in another embodiment of the present invention, a fluid thickness control plate regulates and evens the thickness of the dripped resin fluid. Therefore, it is possible to improve the dimensional precision of the respective cured layers laminated in an integrated manner, so that the dimensional precision of the microchip to be formed is improved.

In addition, since the fluid layers are laminated and cured step by step, a microstructure in a complicated shape can be formed in a high precision.

Yet in another embodiment of the present invention, the non-irradiation area formed in the upper or lower substrate is not cured. Therefore, the resin fluid in the intermediate section can be drained via the three-dimensional spaces in the upper or lower substrate. The upper or lower substrate is formed after the resin fluid is drained. It is possible to utilize the three-dimensional spaces in the upper or lower substrate as an inlet or outlet to supply or drain fluids and gases to or from the three-dimensional spaces.

### BRIEF DESCRIPTION OF THE DRAWINGS

Hereinafter, the microchips regarding the present invention and the manufacturing method of them are described with reference to diagrams.

FIG. 1 is a diagram showing a microchip of the present invention.

FIG. 2 is a flowchart of a microchip manufacturing method of the present invention.

FIG. 3 is a diagram showing the major components of a device used in the microchip manufacturing method of the present invention.

FIG. 4 is a flowchart of manufacturing lower substrate in the microchip manufacturing method of the present invention.

FIG. 5 is a drawing showing a section of manufacturing lower substrate in the microchip manufacturing method of the present invention.

FIG. 6 is a drawing showing a section of manufacturing lower substrate in the microchip manufacturing method of the present invention.

FIG. 7 is a drawing showing the relation between a micromirror array and an area irradiated with light.

FIG. 8 is a drawing that shows forming the microstructure of the microchip.

FIG. 9 is a drawing showing a microchip formed as a cell detecting chip.

FIG. 10 is a perspective view showing a microchip formed as a chip in a cell array type.

FIG. 11 is a drawing showing the lower substrate, intermediate section, and upper substrate of the microchip in FIG. 10.

FIG. 12 is a cross-sectional view of the microchip shown in FIG. 10.

FIG. 13 is a perspective view showing a microchip formed as a micro-flow-channel chip in the lamination structure.

FIG. 14 is a drawing showing the internal structure of the microchip shown in FIG. 13.

FIG. 15 is an exploded view of the microchip shown in FIG. 13.

FIG. 16 is a perspective view of a microchip formed as a micro-flow-channel chip in a damming structure.

FIG. 17 is a drawing showing the internal structure of the microchip shown in FIG. 16.

FIG. 18 is a drawing of one of the walls configuring the intermediate section of the microchip shown in FIG. 16.

FIG. 19 is an expansion perspective view of an incubator used together with the microchip shown in FIG. 16.

FIG. 20 is a cross-sectional view of the assembly of the incubator shown in FIG. 19.

FIG. 21 is a drawing showing the internal state of the microchip when culture media containing cells are entered into the incubator shown in FIG. 19.

FIG. 22 is a perspective view showing a microchip formed as a multi-series microcapillary chip.

FIG. 23 is a drawing showing the microcapillary of the microchip shown in FIG. 22.

FIG. 24 is a drawing showing how to use of the microchip shown in FIG. 22.

FIG. 25 is a drawing showing the first step for selectively taking specific cells, using the microchip shown in FIG. 22.

FIG. 26 is a drawing showing the second step for selectively taking cells using the microchip shown in FIG. 22.

### DETAILED DESCRIPTIONS

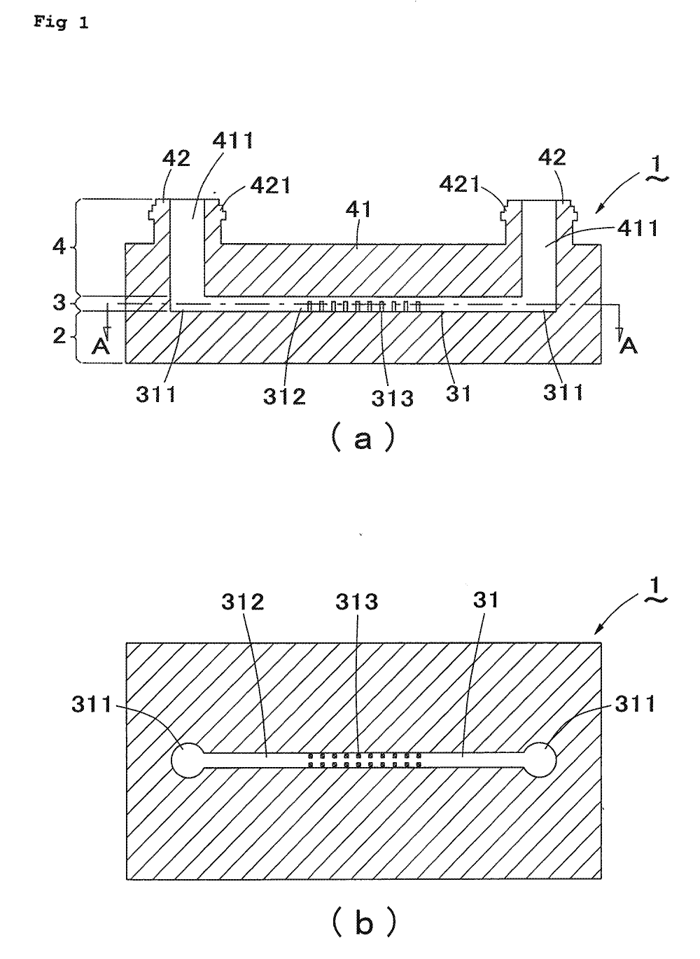

FIG. 1 shows the microchip of the present invention. FIG. 1(a) is a vertical cross-sectional view of the microchip. FIG. 1(b) is a cross-sectional view of FIG. 1(a) along the line A-A. The microchip shown in FIG. 1 is just an example, and other shapes are also incorporated in the present invention.

The microchip (1) is divided into three areas. These three areas are, from the bottom, the lower substrate (2), intermediate section formed on the lower substrate (2), and the upper substrate (4) positioned on the intermediate section (3).

All of the lower substrate (2), intermediate section (3), and upper substrate (4) are made of a light-cured resin, and formed in an integrated manner.

The lower substrate (2) is configured to be a flat plate.

The intermediate section (3) is formed on, and integrated with the top face of the lower substrate (2). The intermediate section (3) includes a cavity (31). In the example shown in FIG. 1, the cavity (31) comprises a pair of substantially cylindrical spaces (311) and a flow channel (312) connecting with these cylindrical spaces (311). The shape and dimensions of the cavity (31) are preferably determined depending on application of the microchip (1). To detect chemical reactions and operate proteins and cells, it is preferable that the cavity (31) has a width and a depth of 10 to 1000 μm.

In the flow channel (312), multiple microstructures (313) are formed. The microstructure (313) protrudes upward from a wall face of the flow channel (312), as well as is integrated with the wall face.

In the example shown in FIG. 1, the microstructure (313) is shaped as a rectangular solid. In addition, multiple microstructures (313) are aligned in the direction of the axis of the flow channel (312), so that they form a pair of lines. For example, when a suspending fluid containing proteins and cells is flown into the flow channel (312), the interval between the lines can be made narrower than the diameter of the protein, so that proteins or cells among the microstructures (313) are captured.

The shape, arrangement, or array of the microstructures (313) is not particularly limited, but determined preferably according to the purposes to which the microchip (1) is applied. It is preferable that the microstructure (313) has a size in a precision of 100 to 100000 nm according to the size of the protein or cell. For example, to use as a scaffold for cell culturing, it is preferable that the microchip has microstructures having a mesh with a size of several tens of micrometers. On the other hand, to separate a DNA equivalent to several tens of thousands to million bases by the electrophoresis method, it is preferable that the microchip has microstructures with a size of 100 to 100000 nm.

The upper substrate (4) is formed on, and integrated with the top face of the intermediate section (3). The upper substrate (4) comprises a substrate portion (41) in a flat plate and cylindrical ducts (42) extending upward from the top face of the substrate (41).

A flow channel whose cross-section is circular (411) is formed inside the duct (42). The flow channel (411) extends along the duct (42), goes through the substrate (41), and connects with the cylindrical space (311) of the intermediate section (3). The flow channel (411) is used as an inlet or outlet to supply or drain fluids or gases to or from the flow channel (312) of the intermediate section (3).

A step portion (421) is formed on the periphery of the top end of the duct (42). The step portion (421) is configured to connect a fluid supplying tool such as a tube with the duct (42).

The shape of the upper substrate (4) is not particularly limited, but determined preferably according to the purposes to which the microchip (1) is applied.

A manufacturing method of the microchip (1) is shown as below. An example of the microchip (1) shown in FIG. 1. is applied in the method.

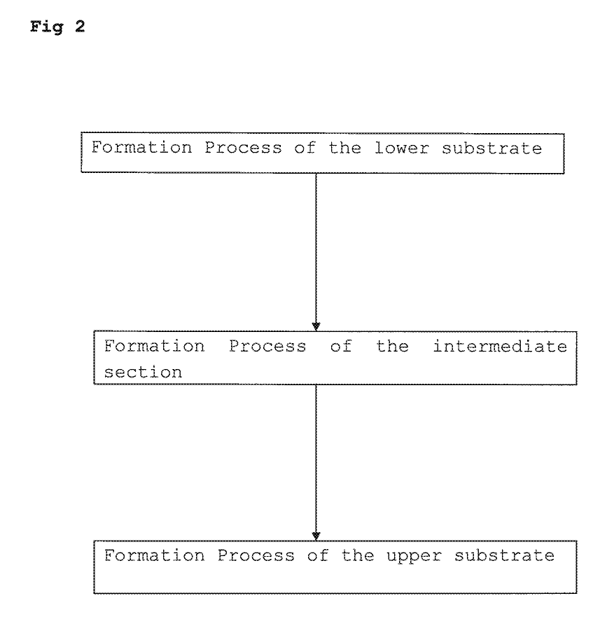

FIG. 2 is a flowchart indicating the manufacturing method of the microchip (1).

The manufacturing method of the microchip (1) comprises formation process of the lower substrate, formation process of the intermediate section, and formation process of upper substrate.

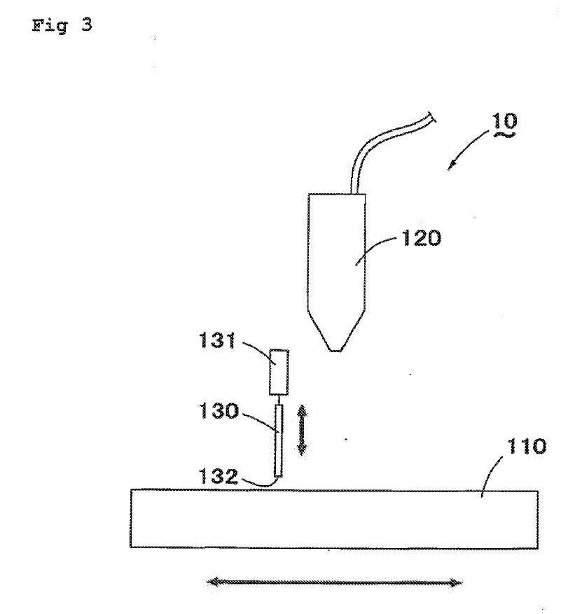

FIG. 3 is a schematic view showing the major portions of the microchip manufacturing device (10) employed in formation process of the lower substrate, intermediate section, and upper substrate.

The microchip manufacturing device (10) is equipped with a stage with a smooth top face (110), a probe (120) which is set above the stage (110) and drips a fluid of a light-cured resin, and a fluid thickness control plate (130) in a thin plate which is set above the stage (110) and has a bottom end (132). The bottom end (132) is parallel to the top face of the stage (110).

The stage (110) is movable in the horizontal direction. The probe (120) is connected to a tank (not shown in the figure) storing a light-cured resin fluid, as well as drips the light-cured resin fluid supplied from the tank onto the top face of the stage (110).

The fluid thickness control plate (130) is connected to a piezoactuator (131). The interval between the bottom end of the fluid thickness control plate (131) and top face of the stage (110) is adjustable in a high precision.

The fluid thickness control plate (130) may be movable not only in vertical direction but also in horizontal direction in stead of horizontal movement of the stage (110) so as to generate relative movement between the fluid thickness control plate (130) and the stage (110).

In addition, although a piezoactuator (131) is connected to the fluid thickness control plate (130) in the above example, it is also allowed to attach an actuator to the stage (110) to make the stage (110) moveable vertically.

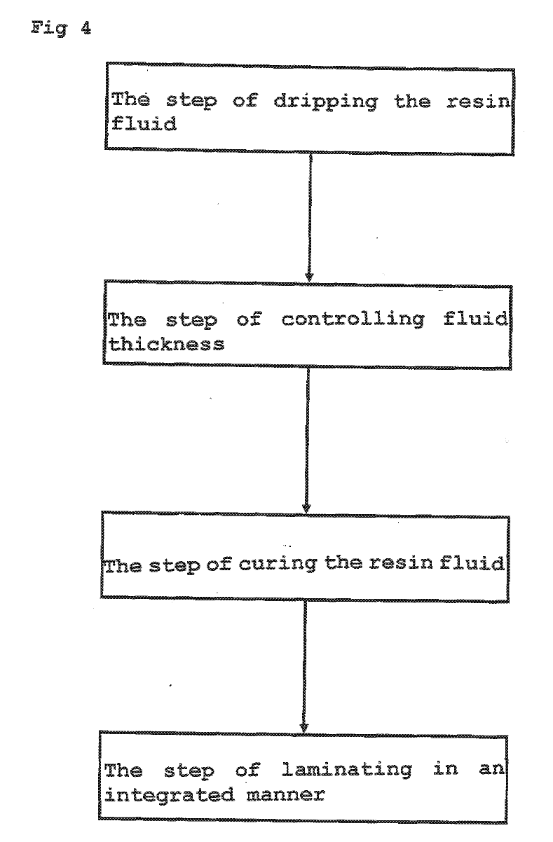

FIG. 4 is a flowchart to show each step of the lower substrate formation process.

The process comprises the step of dripping the resin fluid, controlling fluid thickness, curing the resin fluid, and laminating in an integrated manner.

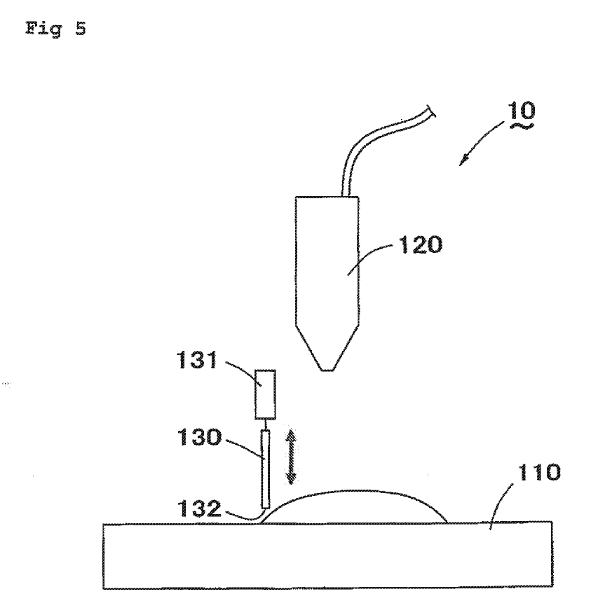

FIG. 5 shows the state after a light-curing resin fluid is dripped onto the stage in the step of dripping the resin fluid.

A light-curing resin fluid is dripped from the probe (120) onto the top face of the stage (110). In this state, the surface of the resin fluid on the stage (110) appears to have round surface due to surface tension.

After the resin fluid is dripped, the piezoactuator (131) is activated to control the interval between the top face of the stage (110) and the bottom end (132) of the fluid thickness control plate (130).

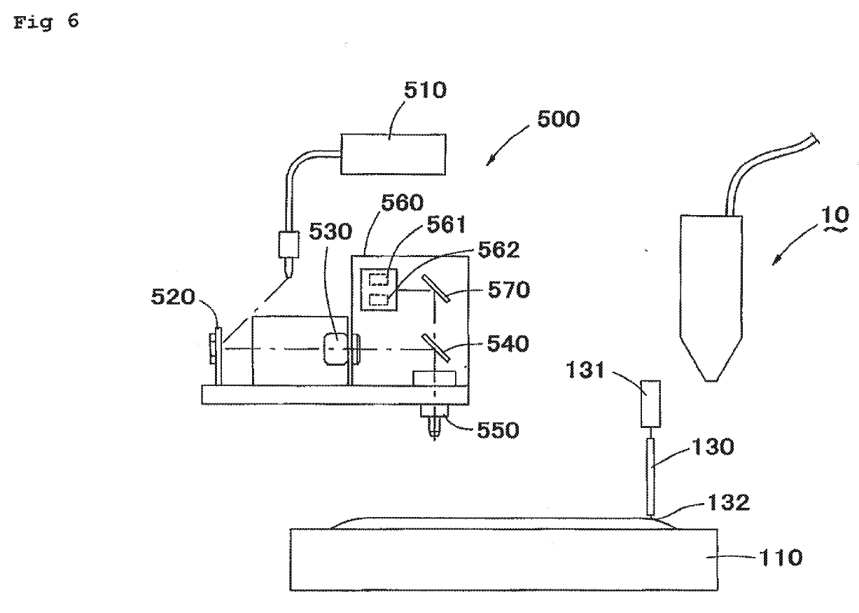

FIG. 6 shows the motions of the stage (110) in the step of controlling the fluid thickness.

Then, the stage (110) is moved horizontally, so that the bottom end (132) of the fluid thickness control plate (130) contacts with the resin fluid. The resin fluid passes through below the bottom end (132).

As shown in FIG. 6, the surface of the resin fluid after passing through below the bottom end (132) is flattened, so that a layer of the resin fluid with an even fluid thickness is formed on the stage (110). The fluid thickness of the resin fluid layer formed by this method is, for example, 1-50 μm, preferably 2-10 μm, and, more preferably 5-10 μm.

In the step of controlling fluid thickness, a layer of a resin fluid with an even fluid thickness is formed on the stage (110), and then the step of curing the resin fluid is executed.

In the step of curing the resin fluid, the layer of the resin fluid is irradiated with light after the fluid thickness is controlled. This cures the light-curing resin in the irradiated area. An optical device for irradiating light (500) is shown in FIG. 6.

The optical device (500) is equipped with a light source (510) and a micromirror array (520) receiving light from the light source (510). In FIG. 6, optical system guiding light from the light source (510) to the micromirror array (520) is not shown.

For the light source (510), for example, it is preferable to apply a semiconductor laser device irradiating light having a wavelength of around 400 nm. For the micromirror array (520), for example, it is preferable to apply DMD (Digital Micromirror Device). Light emitted from the light source (510) is spatially modulated by the micromirror array (520), and is irradiated to the layer of the resin fluid on the stage (110).

Each micromirror in the micromirror array (520) takes various angular positions so that the specific micromirrors in specific angular position can reflect the light. Light from the micromirror array (520) reaches the half mirror (540) via a lens group (530). The light reflected by the half mirror (540) is led to the fluid layer on the stage (110) by an object lens (550).

The optical device (500) is, furthermore, equipped with a detection unit (560) for detecting the interval between the object lens (550) and the face of the fluid layer on the stage (110). The detection unit (560) is equipped with a semiconductor laser device (561) irradiating laser light and an optical receiver (562) receiving reflected light from the top face of the fluid layer.

The laser light emitted by the semiconductor laser device (561) is reflected by a mirror (570), and irradiated to the fluid layer on the stage (110) via the half mirror (540) and object lens (550). This laser light is reflected by the top face of the fluid layer, and is routed to the mirror (570) via the object lens (550) and half mirror (540). Then, the laser light is redirected by the mirror (570), and is received by the optical receiver (562). Here, the optical receiver (562) detects the position where the light is received. This positioning of the optical receiver (562) enables to detect the interval between the object lens (550) and the top face of the fluid layer.

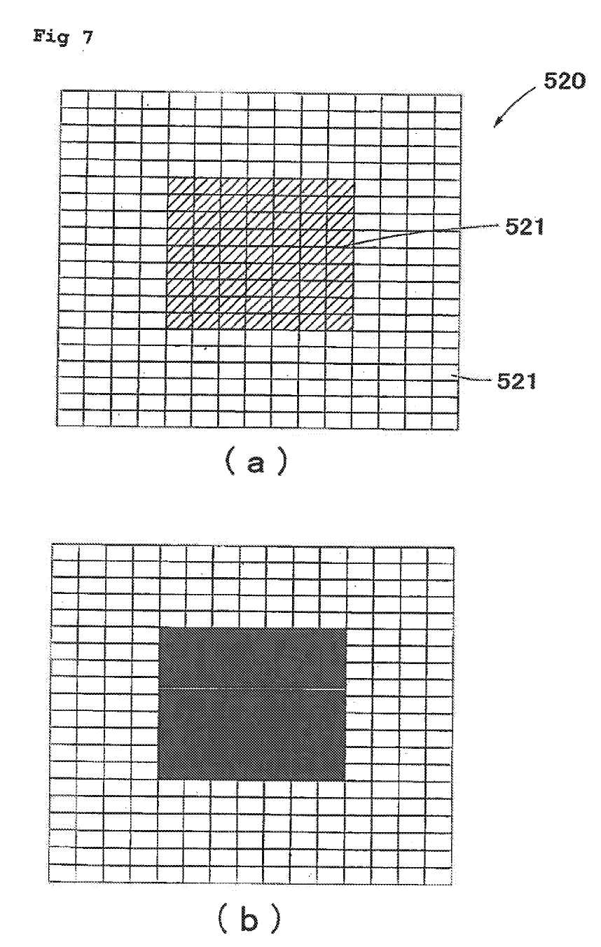

FIG. 7 shows the relation between the motions of the micromirror array (520) and the area irradiated with the light toward the top of the stage (110). FIG. 7(a) shows the posture of each micromirror of the micromirror array (520). FIG. 7(b) shows the area irradiated with the light toward the stage (110) by the micromirror array (520) shown in FIG. 7(a).

As shown in FIG. 7(a), multiple micromirrors (521) are arrayed in the row and the column directions on the micromirror array (520). The angular position of each micromirror (521) is variable to different angle corresponding to certain positions. Each micro mirror (521) can take two types of postures. At one posture, so-called as “ON” position, sends light from the light source (510) to the mirror group (530). At the other posture, so-called as “OFF” position, does not send light to the mirror group (530). In the example shown in FIG. 7(a), the micromirror positioning the “ON” position (521) is hatched.

The area irradiated with the light toward the top of the stage (110) can be changed by the posture of the above micromirror (521). Each micromirror (521) may irradiate light to certain positions on the stage (110), and as shown in FIG. 7(b), the area irradiated on the stage (110) is partitioned by latticed areas corresponding to the micromirrors (521).

In the example shown in FIG. 7(a), since light is sent only from the hatched micromirrors (521) to the mirror group (530), only the corresponding latticed areas (blacked out areas) are irradiated. The micromirror array (520) has a rectangular shape, and a side of the pitch of each micromirror is approximately 10 μm long, for example, 13.68 μm long. The interval between a micromirror and an adjacent one is, for example, 1 μm. The whole size of the DMD2 applied in the present embodiment 1 is shaped as a rectangle of 40.8×31.8 mm. (The mirror thereof has a rectangular shape with an area of 14.0×10.5 mm.) DMD2 consists of 786,432 micromirrors, and each micromirror has a 13.68 μm side length.

As mentioned above, the micromirrors (521) are operated to control the area irradiated with light toward the stage (110). Next, a preferred area size, which is the area as the lower substrate (2) in the microchip (1), is irradiated with light, and the light-curing resin is cured. Then, the stage (110) is moved horizontally, and light is irradiated to the fluid of the light-curing resin sequentially. A layer of the cured resin, which is to be the first layer, is formed on the stage (110), and then the step of lamination is proceeded.

At the lamination step, first, the stage (110) is moved below the probe (120), and the probe (120) drips the fluid of the light-curing resin onto the cured resin layer formed as described above.

Next, the piezoactuator (131) is activated to raise the fluid thickness control plate (130). Then, while the bottom end (132) of the fluid thickness control plate (130) contacts the dripped resin fluid, the stage (110) is moved horizontally so that the resin fluid passes through below the bottom end (132).

Then, while the posture of the micromirrors (521) is controlled, light is irradiated to the stage (110), furthermore, the curing resin layer is laminated.

Lower substrate (2) with a certain thickness is formed by repeating the above steps of dripping resin, controlling the fluid thickness, and curing resin. In the step of the lamination, fluid thickness is controlled in each laminating cycle. Therefore, error in the direction of thickness, which is caused by lamination, is not accumulated, so that the lower substrate (2) can be formed in a very high precision.

After the lower substrate (2) is formed, the formation process of intermediate section is proceeded.

Also in the formation process of the intermediate section, the above steps of dripping, controlling fluid thickness, and curing resin are repeated on that lower substrate (2).

As mentioned above, the intermediate section (3) includes with cavities (311) and microstructures (312).

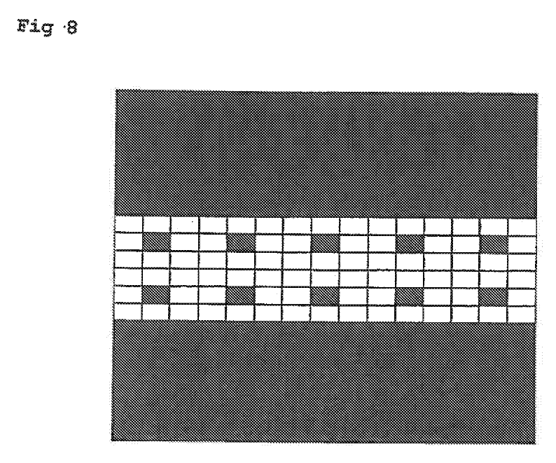

FIG. 8 shows the area irradiated with light toward the stage (110) when the microstructures (312) are formed. As mentioned above, the angular position of the micromirrors (521) is controlled. And the micromirrors (521) corresponding to the positions of the microstructures (312) are set to the “ON” position. The micromirrors (521) corresponding to the flow channel (312) are set to the “OFF” position. Furthermore, the micromirrors (512) corresponding to the outside area of the flow channel (312) are set to the “ON” position. Then, the light is irradiated from the light source (510). The fluid of the light-curing resin at the position corresponding to the micromirrors (521) positioned at the “ON” position is cured.

In the case of forming a microstructure (312) with a complicated cross-sectional shape, it is only necessary to reduce the size of the micromirror (521) and form a micromirror array (520) having more micromirrors (521).

In addition, in the case of forming a microstructure (312) with complicated shape variations in the thickness direction, the raise of the fluid thickness control plate (130) is reduced, so that a thin resin fluid layer is formed. And curing treatment is applied to the thin resin fluid layer. It is possible to achieve high shape precision, by repeating this procedure serially.

After the intermediate section (3) is formed in this way, formation process of the upper substrate is proceeded. Also in the formation process of the upper substrate, the above-mentioned steps of dripping resin, controlling fluid thickness, and curing resin are repeated on the formed-intermediate section (3).

As mentioned above, the upper substrate (4) includes flow channels (411). Consequently, in the same way as the motion performed in the formation process of intermediate section, the areas corresponding to the flow channels (411) are not irradiated with light, and hereinafter referred as non-irradiation areas. Areas other than the flow channels (411) are irradiated with light, and hereinafter referred as irradiation areas. Lamination is performed by repeating the steps of dripping resin, controlling fluid thickness, and curing resin. Consequently, the resin fluid in the three-dimensional space consisting of non-irradiation areas is not cured, but kept in the liquid phase. Resin fluid in the other areas is cured, so that the upper substrate (4) is formed.

The ducts (42) are formed by light irradiation to the resin fluid where the walls of the ducts (42) are configured, and by leaving the other portions as non-irradiation areas.

Although the flow channels (411) are formed in the upper substrate (4) in the above example, it is also possible to form a flow channel to connect with the cavity (31) of the intermediate section (3) inside the lower substrate (2).

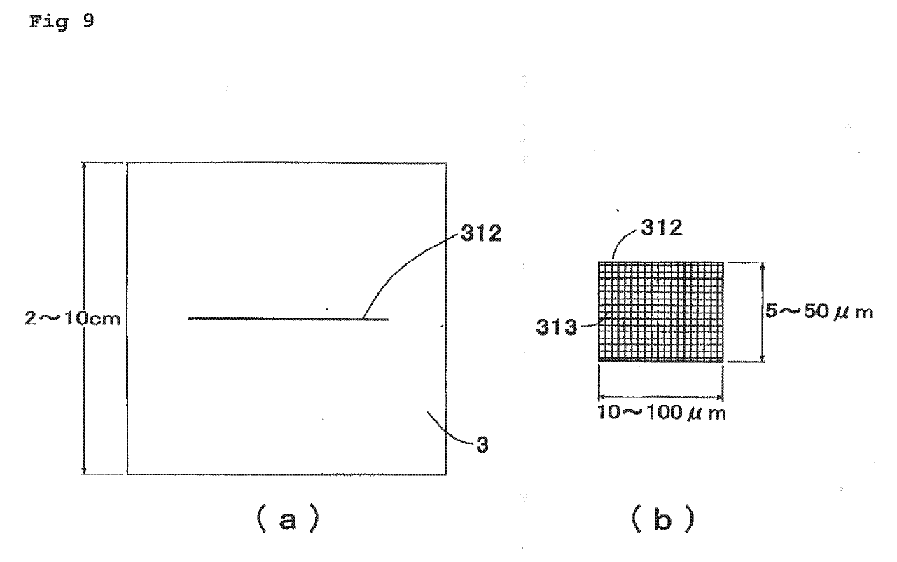

In this way, in the present invention, various shapes from a very large structure to a very minute structure can be formed in a high precision. FIG. 9 show the microchip formed as a cell detecting chip obtained by the above method. FIG. 9(a) is a cross-sectional view of a microchip (1) at the intermediate section (3), showing the contrast of size between the cavity (312) formed in the intermediate section (3) and the whole microchip. FIG. 9(b) is an extended view of the cross-section of the cavity (312).

Using the above method, it is possible to form a microchip with a side of several centimeters long (for example, 2 to around 10 centimeters long), and form a groove (cavity) (31) with a width of 10 to 1000 μm and a depth of 5 to 100 μm in the intermediate section (3). Then, at a cross-section in the groove (31), a latticed structure can be formed as microstructures (313). The meshes of this latticed structure can be formed as rectangles with a side of 100 to 100000 nm long.

For example, it is possible to detect an influenza virus having a DNA of several tens of thousands to several million bases without using any gels or polymer solutions, by making a suspending fluid containing cells into such the cavity.

In addition to the above structure, it is also possible to form a microcavity (31) and use the microchip as a cell chip to culture single cells in the cavity. Culturing single cells in the microcavity (31) enables to analyze cell functions with small quantity of culture media and a small number of cells. In addition, since such a microcavity (31) is used as a cell culturing tank, it is not required to concentrate and separate metabolites of cells from the culture media. Therefore, cell functions can be analyzed in a real-time manner.

Moreover, the flow speed of the fluid is reduced in the groove of the fluid (31) by introducing two types or more types of reagents or gases into the microgroove (31). Therefore, such reagents or gases may be blended depending on a molecular diffusion to eliminate the need of mechanical stirring. In addition, this also results in efficiently facilitating the solid-liquid interface reaction and liquid-liquid interface reaction on the inner wall of the groove (31). And, this also results in enhancing the reaction speed, when extraneous heating and cooling are performed.

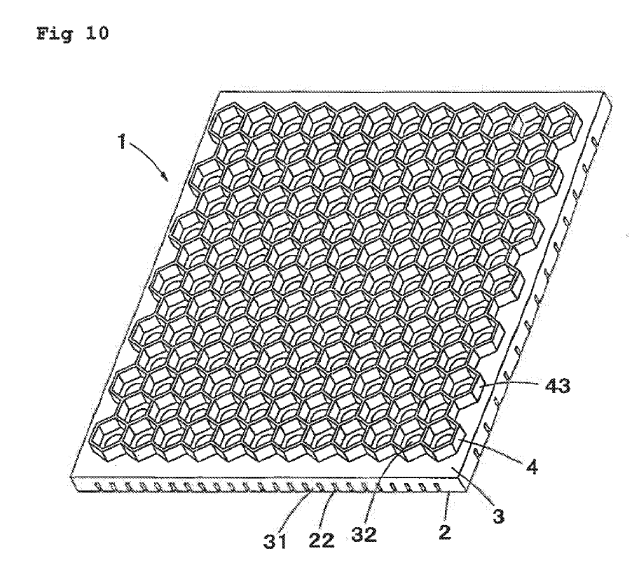

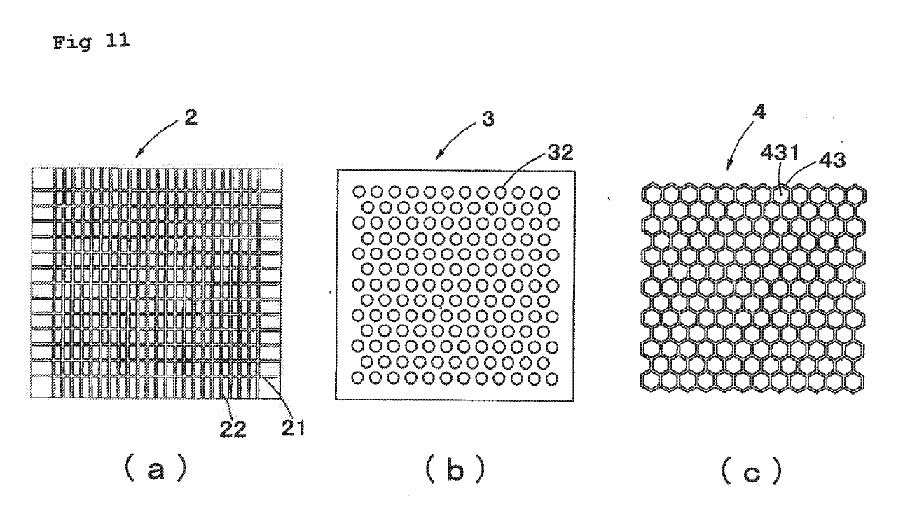

FIG. 10 is a perspective view to show a microchip (1) formed as a chip of the cell array type, which can be obtained by the above method. FIG. 11 is an exploded view to show the lower substrate (2), intermediate section (3), and upper substrate (4) of the microchip (1) shown in FIG. 10. FIG. 11(a) is a plan view of the lower substrate (2) of the microchip (1) shown in FIG. 10. FIG. 11(b) is a plan view of the intermediate section (3) of the microchip (1) shown in FIG. 10. FIG. 11(c) is a plan view of the upper substrate (4) of the microchip (1) shown in FIG. 10.

In the formation process of lower substrate, an array of small rectangle blocks (21) is formed. The whole lower substrate (2), which is an array of the rectangle blocks (21), is formed like a rectangular flat plate on a plan view.

The rectangle blocks (21) are arrayed like a matrix, and grooves (22) extending vertically and horizontally are formed among the rectangle blocks (21). The grooves (22) form a lattice pattern in the lower substrate (2).

It is preferable that the groove (22) has a width of 1 to around 50 μm. In addition, it is preferable that the lower substrate (2) has a thickness of 10 to around 100 μm. These formations enable the culture media to flow into the grooves (22).

In the formation process of intermediate section, an intermediate section (3) is integrally laminated on the top face of the lower substrate (2) formed as mentioned above. The contour of the intermediate section (3) is formed in the same shape and size as the lower substrate (2).

The intermediate section (3) includes multiple circular apertures (32). The circular apertures (32) are arrayed so that the centers of the circular apertures (32) match the intersections between the vertical grooves (22) and horizontal grooves (22) in the lower substrate (2). If the diameter of the circular aperture (32) is 10 to around 300 μm, cells can be contained in it preferably.

Formation process of the intermediate section (3) enables to connect each rectangle block (21) configuring the lower substrate (2).

In the formation process of the upper substrate, the upper substrate (4) is integrally formed on the top face of the intermediate section (3) formed as mentioned above.

The upper substrate (4) consists of walls (43) like thin plates protruding upward from the top face of the intermediate section (3). The walls (43) are connected each other to form right hexagonal areas (431) on a plan view. Those hexagonal areas (431) are adjacent to each other across the walls (43), so that the whole upper substrate (4) is shaped as the honeycomb structure.

The centers of the areas (431) match those of the circular apertures (32) formed on the intermediate sections (3).

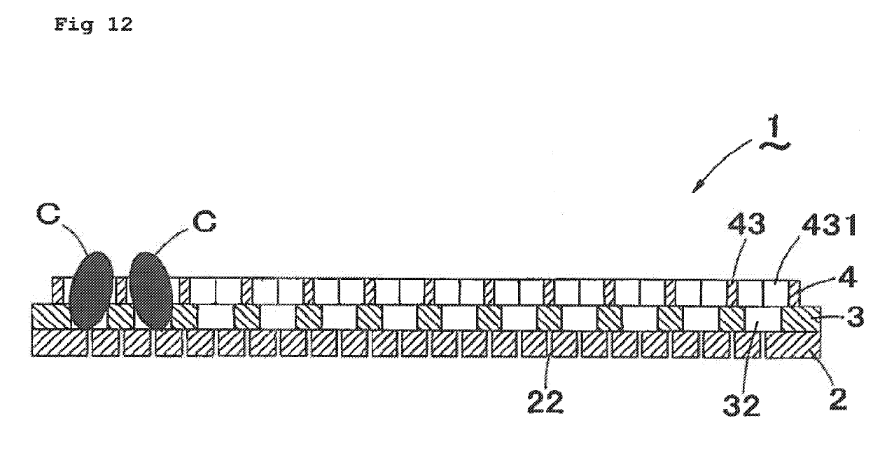

FIG. 12 is a cross-sectional view of the microchip (1) shown in FIG. 10, showing how to use the microchip (1) in FIG. 10.

The circular aperture (32) formed in the intermediate section (3) and the honeycomb areas (431) in the upper substrate (4) are connected each other to form a cell containing space. Cells (C) are contained in the cell containing spaces.

Culture media flow into the grooves (22). The culture media reach the cells (C) through the circular apertures (32), and supply nutrients required for cell cultivation. Moreover, the culture media go through the grooves (22), so that waste products from the cells (C) and aged culture media are drained out of the microchip (1).

Using the microchip (1) in FIGS. 10 to 12 enables to culture the regularly-arranged cells. The culturing cell bonds the neighboring cells each other, so that they function as a cellular organization. This cellular organization has a structure more approximate to in-vivo cellular organizations than to those cultured on a substrate randomly. This makes it possible to form a cell chip with improved functionality as a cellular organization.

A through hole can be created on the wall (43) of the upper substrate (4) so that the mutual bonding of neighboring cells is facilitated. It is also possible to culture cells with piling up multiple pieces of the microchip (1) in FIGS. 10 to 12.

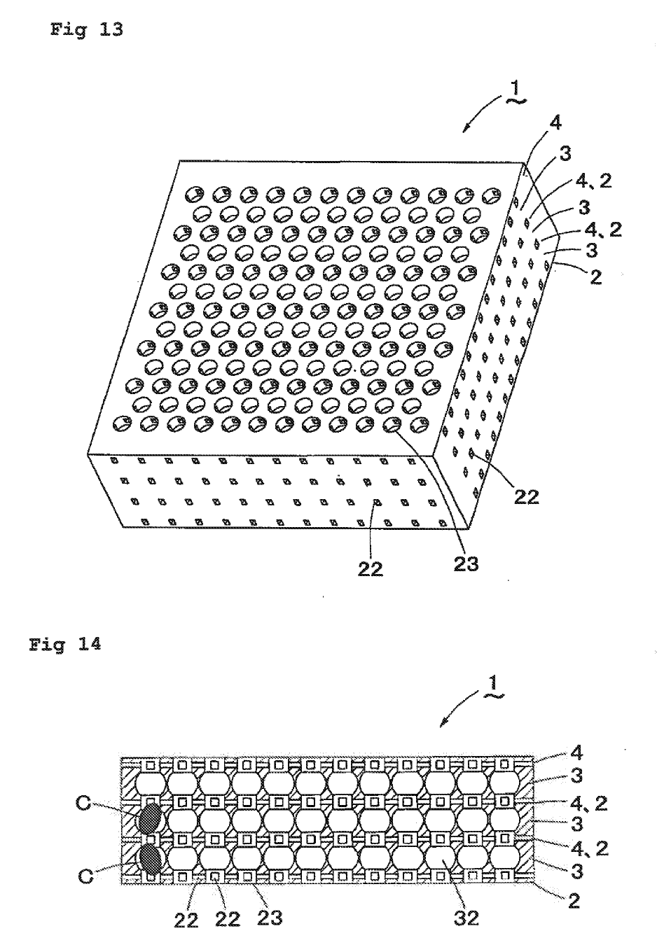

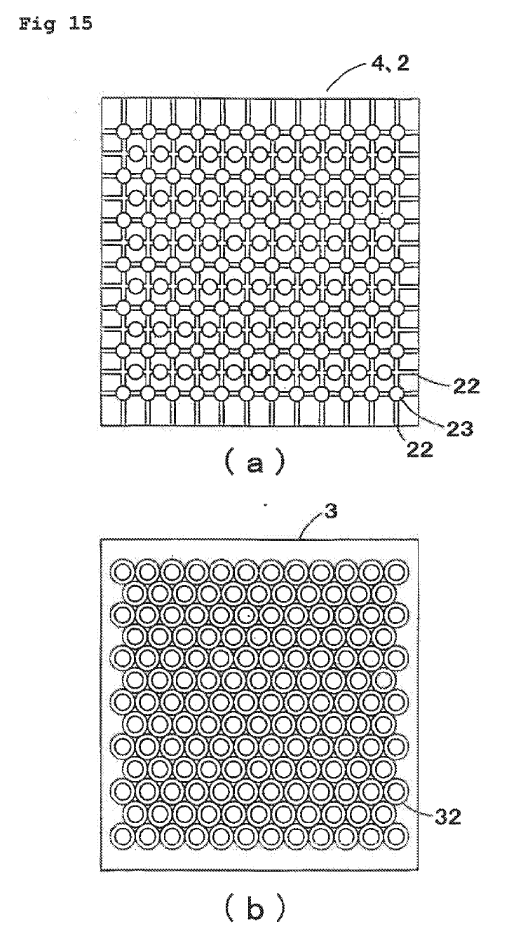

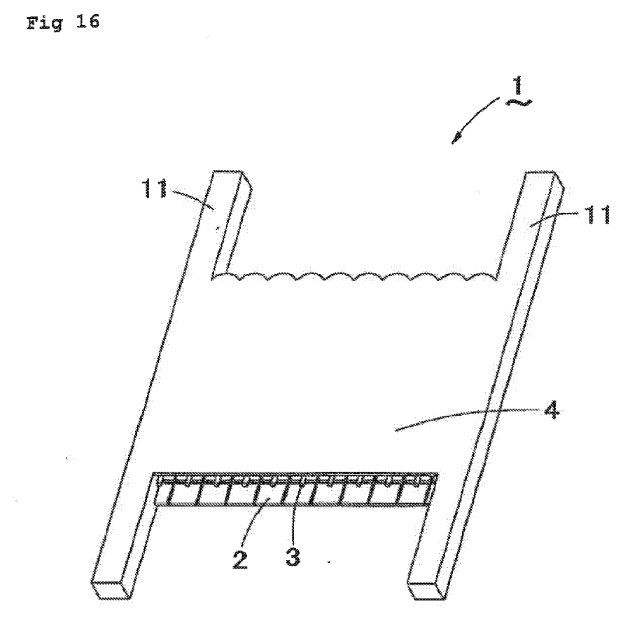

FIG. 13 is a perspective view showing a microchip (1) formed as a chip to array cells in lamination by the above method. FIG. 14 is a vertical cross-sectional view of the microchip (1) shown in FIG. 13, and FIG. 15 is an exploded plan view of each layer configuring the microchip (1) shown in FIG. 13. FIG. 15(a) is a plan view of the lower substrate (2) and upper substrate (4), and FIG. 15(b) is a plan view of the intermediate section (3).

As shown in FIGS. 13 and 14, the microchip (1) includes a lower substrate (2), an intermediate section (3) formed on the top face of the lower substrate (2), and an upper substrate (4) formed on the top face of the intermediate section (3).

As shown in FIG. 15, in the present embodiment, the lower substrate (2) and upper substrate (4) have the same shape.

The upper substrate (4) at the middle position of the microchip (1) in the thickness direction has a function as a lower substrate (2). An additional intermediate section (3) is formed on the top face of this upper substrate (4). Furthermore, an upper substrate (4) is formed on the top face of this intermediate section (3).

In this way, the microchip (1) has a lamination structure.

Then, the structures of the lower substrate (2), upper substrate (4), and intermediate section (3) are described.

In formation process of the lower substrate, an array of the circular apertures (23) and grooves (22) are formed. The circular apertures (23) are arrayed like a matrix. Then, the grooves (22) extending vertically and horizontally are formed, so that the circular apertures (23) connect with the grooves. The grooves (22) form a lattice pattern in the lower substrate (2).

It is preferable that the width of the groove (22) is 1 to around 50 μm. In addition, it is preferable that the thickness of the lower substrate (2) is 10 to around 100 μm. This formation enables to move the culture media into the groove (22) preferably.

In the formation process of the intermediate section, an intermediate section (3) is integrally laminated on the top face of the lower substrate (2) formed as mentioned above. The contour of the intermediate section (3) is formed in the same size and shape as the lower substrate (2).

The intermediate section (3) includes multiple globular cavity apertures (32). The globular cavity apertures (32) are arrayed so that the centers of those apertures (32) match the centers of the circular aperture (23) located at the intersections between the vertical grooves (22) in the lower substrate (2) and the horizontal grooves (22) in the lower substrate (2). If the diameter of the globular cavity aperture (32) is 10 to around 300 μm, cells can be contained therein preferably.

Upper substrate (4) has the same shape as the fore mentioned lower substrate (2).

An upper substrate (4) is integrally laminated again on the top face of the intermediate section (3). The upper substrate (4) functions as a lower substrate (2). An intermediate section (3) is additionally laminated on this upper substrate (4). The repeating lamination enables multilayer arrangement of the cells.

As shown in FIG. 15, the circular apertures (23) formed on the lower substrate (2) and upper substrate (4) are connected with the globular cavity apertures (32) formed in the intermediate section (3). Cells (C) are contained in the space where those circular apertures (23) and cavity apertures (32) are connected. As mentioned above, the microchip (1) has the lamination structure, so that laminated spaces for containing cells are formed in the microchip (1).

Culture media flow into the grooves (22). The flowed culture media reach the cells (C) contained in the globular cavity apertures (32) connected with the circular apertures (23), and the flowed culture media supply the nutrients necessary for culturing cells. Moreover, the culture media go through the grooves (22), so that waste products from the cells (C) and aged culture media are drained from the microchip (1).

Culturing cells in a lamination structure enables to create a cell chip with organ functions, and a microorgan for transplant.

FIG. 16 is a perspective view showing a microchip (1) formed as a micro-flow-channel chip with a damming structure obtained by the above method.

The microchip (1) shown in FIG. 16 includes a pair of arms (11) on the left and right sides to stabilize flowing of the fluid into the microchip (1). The arms (11) extend in the direction of fluid's flow. Those arms (11) are arrayed parallel to one another.

At the intermediate of the arms (11), a lower substrate (2) extends from the bottom ends of the arms (11) between a pair of arms (11). An upper substrate (4) extends from the top end of the arms (11) between a pair of arms (11).

An intermediate section (3) is arranged between the lower substrate (2) and upper substrate (4).

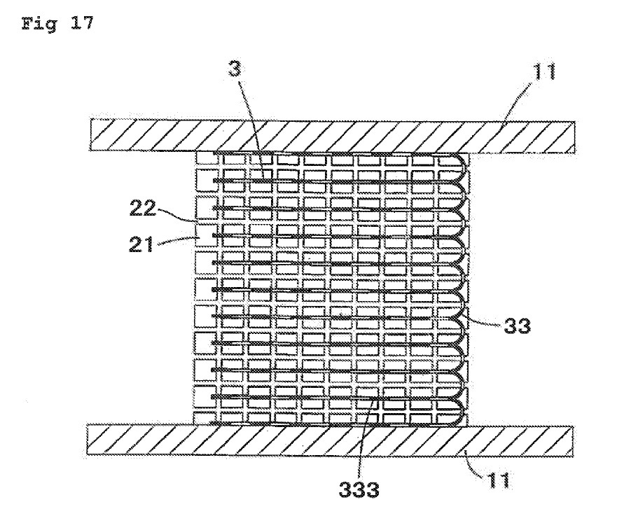

FIG. 17 is a perspective view showing the microchip (1) in FIG. 16 without the upper substrate (4). Thus FIG. 17 shows the structures of the lower substrate (2) and intermediate section (3).

In the formation process of the lower substrate (2), an array of small rectangle blocks (21) is formed. The whole lower substrate (2), which is an array of rectangle blocks (21), is formed like a rectangular flat plate on a plan view.

Rectangle blocks (21) are arrayed like a matrix, and grooves (22) extending vertically and horizontally are formed among the rectangle blocks (21). The grooves (22) form a lattice pattern in the lower substrate (2).

It is preferable that the groove (22) has a width of 1 to around 50 μm. In addition, it is preferable that the lower substrate (2) has a thickness of 10 to around 100 μm. The formation can move culture media into the groove (22) preferably.

The rectangle blocks (21) adjacent to the arms (11) are integrated with the arms (11).

In the formation process of the intermediate section, the intermediate section (3) is integrally laminated on the top face of the lower substrate (2) formed as mentioned above.

The intermediate section (3) consists of multiple U-shaped thin walls (33) on a plan view, and the multiple walls (33) are connected with each other in the width direction of the flow channel. The linear portion (331) of the walls (33) crosses the centers of the rectangle blocks (21) arrayed in the flow direction of the fluid to connect those blocks (21) with each other. The carved portion (332) of the wall (33) connects the rectangular blocks arranged closest to the downstream of the lower substrate (2) with each other. Consequently, the all rectangle blocks (21) configuring the lower substrate (2) are connected each other.

The wall (33) adjacent to a pair of arms (11) are integrated with the inner walls of the arms (11).

The upper substrate (4) is configured to cover the U-space at the top of the intermediate section (3) formed as described above.

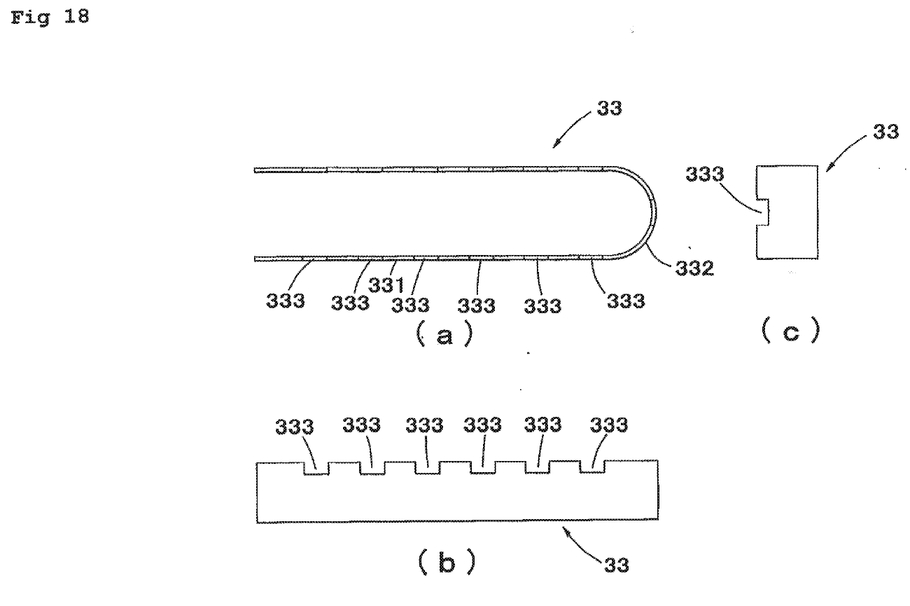

FIG. 18 is a schematic view of one of the multiple walls (33) configuring the intermediate section (3). FIG. 18(a) is a plan view of the wall (33). FIG. 18(b) is a front view of the wall (33). FIG. 18(c) is a view of the wall (33) from the downstream side.

Rectangular notches (333) are formed on the top end of the linear portion (331) and the carved portion (332) of the walls. The notch formed on the linear portion creates a flow of fluid crossing the U-space partitioned by the wall (33). In addition, the notch (333) formed at the carved portion (332) makes it possible to drain the fluid flowed into the U-space.

The notch (333) is formed in the smaller size, compared to the size of the cell or particle suspended in the flowing fluid.

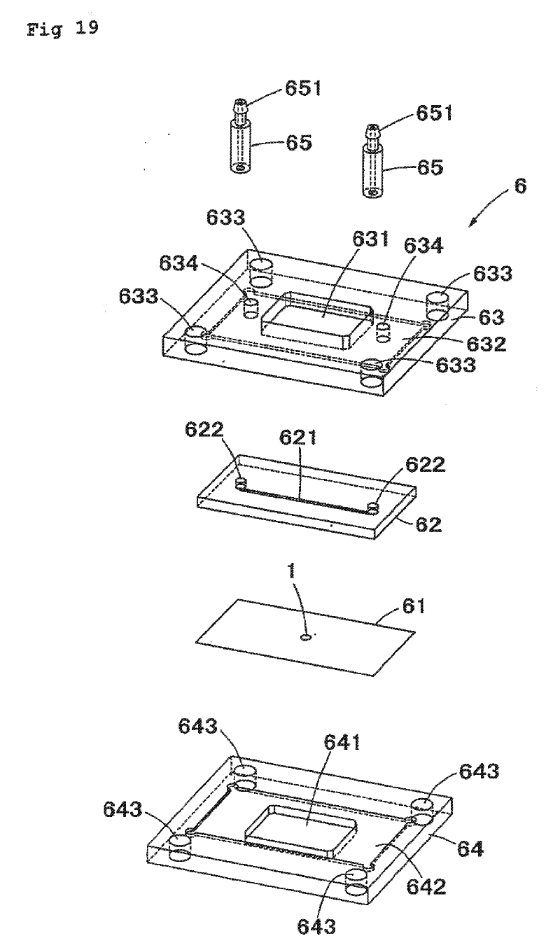

FIG. 19 is an exploded perspective view of the incubator employed together with the microchip (1) shown in FIGS. 16 to 18. Each member shown in FIG. 19 is made of a light-transmissive material, and suitable for observation with an optical microscope.

The incubator (6) comprises a chip substrate (61), a flow channel forming plate (62) fixed on the top face of the chip substrate (61), an upper clamping plate (63) and lower clamping plate (64) for clamping the chip substrate (61) and flow channel forming member (62) from upside and downside, and a pair of tube connectors (65) connected with the upper clamping plate (63).

The microchip (1) shown in FIGS. 16 to 18 is fixed on the top face of the chip substrate (61). A thin flow channel (621) is formed on the bottom face of the flow channel forming plate (62). Putting the flow channel forming plate (62) on the chip substrate (61) makes the bottom of the flow channel (621) sealed on the chip substrate (61). When the flow channel forming plate (62) is piled up on the chip substrate (61), the microchip (1) is present in the flow channel (621). Here, the arms (11) of the microchip (1) are set parallel to the axis of the flow channel (621).

The flow channel forming plate (62) includes a pair of through-holes (622). Each of the pair of through-holes (622) is connected with each end of the flow channel (621).

Both the upper clamping plate (63) and lower clamping plate (64) are members shaped in flat plates. Rectangular openings (631, 641) are formed at the centers of the upper clamping plate (63) and lower clamping plate (64), so that preferable observation with the optical microscope is performed. When the incubator (6) is assembled, the microchip (1) is located at the centers of the openings (631, 641).

Rectangular receptions (632, 642) are formed on the bottom face of the upper clamping plate (63) and the top face of the lower clamping plate (64). When the upper clamping plate (63) is piled on the lower clamping plate (64), the receptions (632, 642) form a room to contain a lamination of the chip substrate (61) and flow channel forming plate (62).

Through-holes (633, 643) are formed at the four corners of the upper clamping plate (63) and lower clamping plate (64), and a fixture such as a bolt is inserted into the through-holes (633, 643). Consequently, the upper clamping plate (63) is tightly contacted to the lower clamping plate (64) to fix the lamination of the chip substrate (61) and flow channel forming plate (62) in the room formed by the receptions (632, 642).

The upper clamping plate (63) includes a pair of through-holes (634). The through-hole (634) of the upper clamping plate (63) is connected with the through-hole (622) formed on the flow channel forming plate (62).

Connectors (65) are inserted into the through-holes (634) on the upper clamping plate (63). Connectors (65) are substantially cylindrical, and include fixing portions (651) at its top ends of the connectors (65) to fix the tubes.

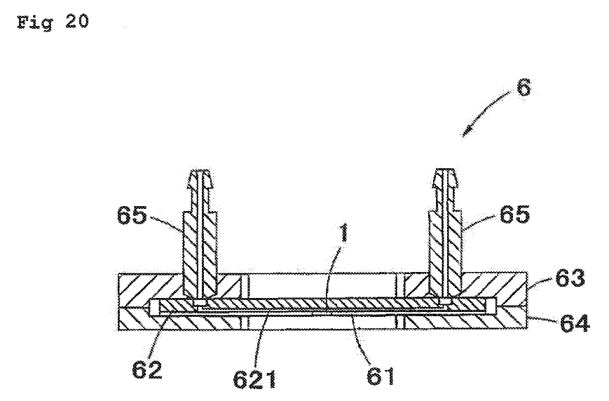

FIG. 20 is a cross-sectional view of the assembly of the incubator (6) shown in FIG. 19.

Fluid is supplied from one of the connectors (65). Particles such as cells are suspended in the supplied liquid. A fluid entering from one of the connectors (65) is drained to the other connector through the flow channel (621).

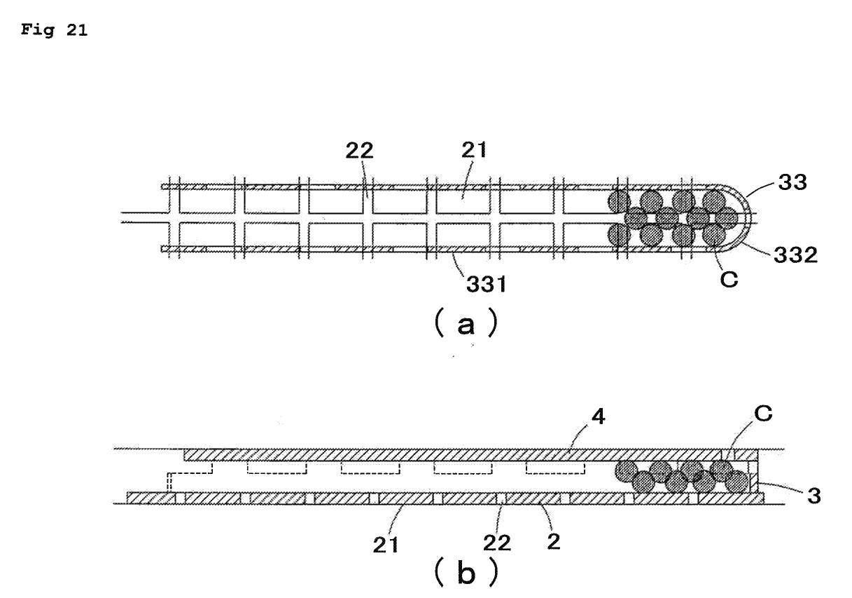

FIG. 21 shows the inside of the U-shape wall (33) in the microchip (1) that is put in the incubator (6) in FIG. 20. FIG. 21(a) is a plan view of the space enclosed by the wall (33), and FIG. 21(b) is a cross-sectional view of the space enclosed by the wall (33).

The particles (C) in the fluid supplied from the connectors (65) gather at the carved portion (332) of the U-shape wall (33). The space on the upper stream space than the carved portion (332) is formed as a thin space enclosed by the wall (33), lower substrate (2), and upper substrate (4). Therefore, the fluid flows in this space as a moderate laminar flow. Accordingly, the particles (C) stably gather around the carved portion (332).

Dimensions such as the distance of the linear portion (331) of the wall (33), the thickness of the intermediate section (3), or the curvature radius of the carved portion (332) of the wall (33) can be determined according to the average grain diameter of the particles (C) in the supplied fluid. These dimensions are optimized so that the blocked particles (C) have the densest structure. This makes it possible to arrange more particles (C) in a plain without overlapping the particles (c) each other in a microscopic observation. Thus more particles (c) can be observed simultaneously.

Since microchip (1) consists of light-transmissive materials, the gathered particles can be observed using a light microscope. Therefore, it is possible, for example, to stop supplying fluid, when a desired quantity of particles is accumulated.

An example is shown that those particles are cells. If the particles are cells, fluid containing cells may be supplied until gathering a desired amount of cells. And then the fluid supply may be stopped and culture media may be flowed into the flow channel (621). The culture media provide necessary nutrients with the cells gathered at the carved portion (332), as well as wash waste products excreted from the cells toward the downstream. In addition, it is also possible to make agents flow into the flow channel (621) together with the culture media, so that the interaction between the agents and cells are evaluated or analyzed.

As mentioned above, it is possible to make cells gather densely so as to culture or examine those cells.

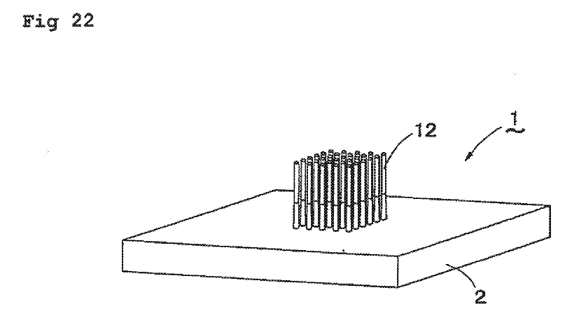

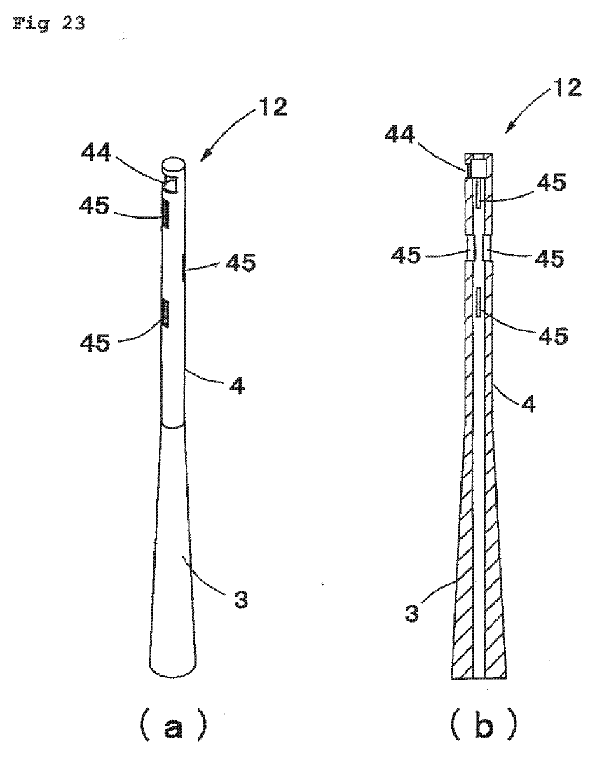

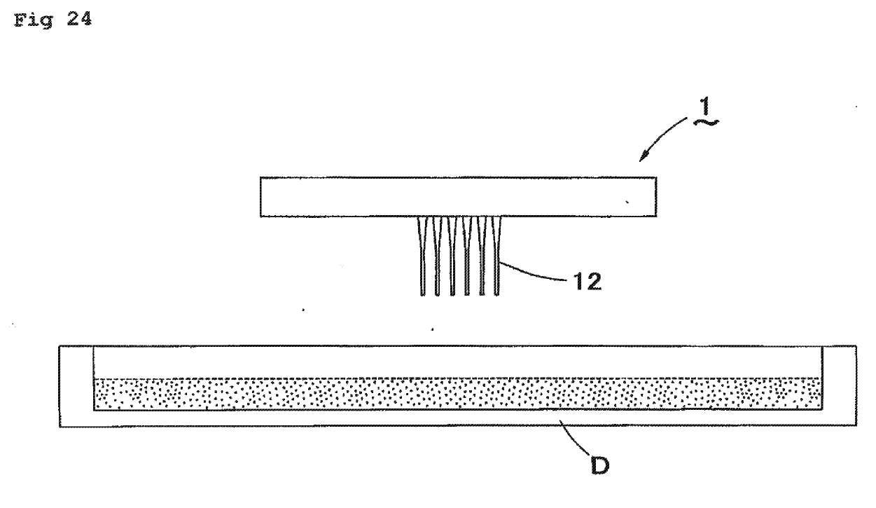

FIG. 22 is a perspective view showing the microchip (1) formed as a multi-series microcapillary chip obtained by the above method. FIG. 23 shows the microcapillaries of the microchip shown in FIG. 22. FIG. 23(a) is a perspective view of a microcapillary, and FIG. 23(b) is a cross-sectional view thereof.

The microchip (1) shown in FIG. 22 consists of the lower substrate shaped in a flat plate (2) and the multiple pieces of microcapillary (12) protruding upward from the top face of the lower substrate (2). The lower substrate (2) is formed in the formation process of the lower substrate. The microcapillary (12) is formed during the formation process of the intermediate section and the formation process of the upper substrate portion are proceeded.

A microcapillary (12) consists of an intermediate section with a trapezoidal cone shape (3) and an upper substrate portion with a cylinder shape (4). The microcapillary (12) is formed in the hollow structure.

A cell introducing hole (44) with rectangular shape is formed on the periphery of the top end of the upper substrate portion (4) to be connected with the internal space of the microcapillary (12). In addition, multiple fluid-introducing holes (45) are formed on the periphery of the upper substrate portion (4) below the cell introducing hole (44).

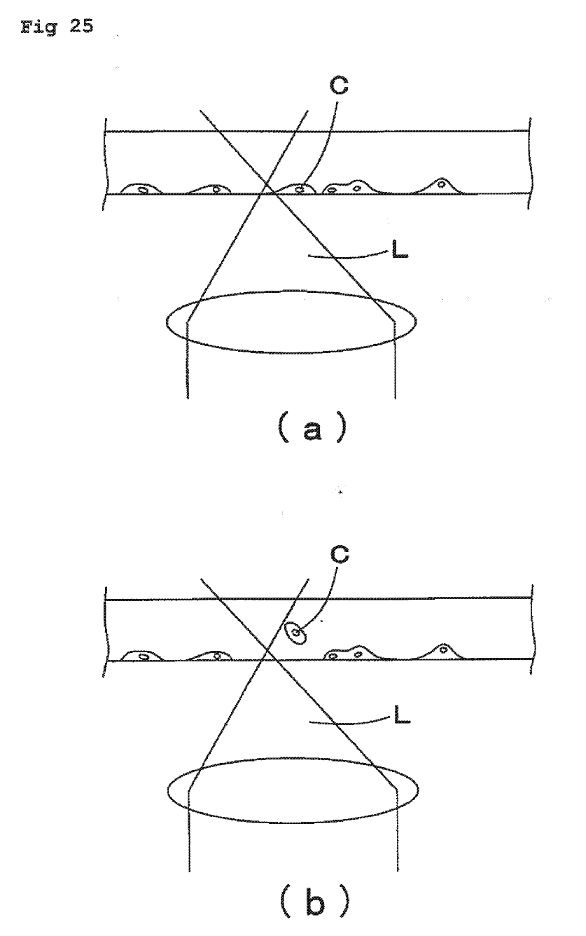

FIG. 24 shows a form how to use the microchip (1) shown in FIGS. 22 and 23.

While using the microchip (1), it is set upside down compared to the state in forming the microchip (1). The microchip (1) is used to selectively take specific cells out of the dish (D) by inserting the microcapillary (12) into culture media containing cells.

FIG. 25 shows the first step of taking the specific cells. FIG. 25(a) shows a condition during the first step. FIG. 25(b) shows a condition after the first stage.

Many of the cells cultured in the dish (D) are bonded to the bottom face of the dish (D). Therefore, it is necessary to peel the cells (C) from the bottom face of the dish (D).

At the first step, the cell (C) is peeled from the bottom face of the dish (D).

First, the target cell to be taken out separately is selected, and the periphery of the selected cell is irradiated with laser light (L). The types of the laser light (L) is not particularly limited, but preferably, UV laser, femtosecond laser, and so on are applicable.

The area around the periphery of the selected cell (C) is cut off by scanning with laser light (L). When femtosecond laser is applied, the laser light (L) is focused around the portion where the cell is cut off by scanning with laser light (L). Then, the intensity of the laser light is controlled for causing a shock wave in the culture media. This shock wave has the cell (C) peeled from the bottom face of the dish (D). When UV laser is used, the cell is cut off by scanning with laser light (L). After the cut is finished, the laser light (L) is focused at the bottom face of the dish, and the bottom face of the dish (D) is destroyed. The destruction makes it possible to peel the cell (C) from the bottom face of the dish (D).

Proceeding the first step operations to multiple cells (C) enables to float those cells (C) in the culture media.

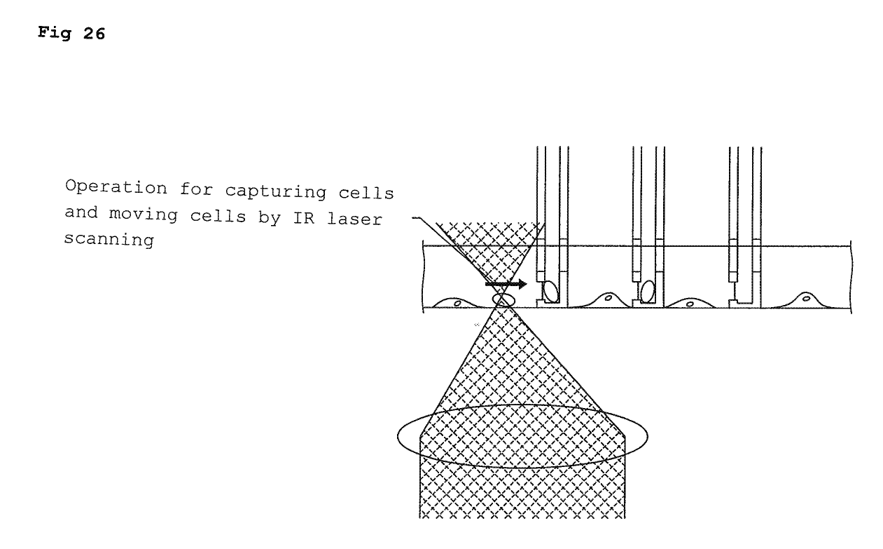

FIG. 26 shows the second step of taking out specific cells.

At the second step, the microcapillaries (12) of the microchip (1) are inserted into culture media. The culture media flow from the fluid introducing hole (45) into the microcapillaries (12).

Cells (C) floating in the culture media are captured by IR laser at the focal point. Then, the focal point of the IR laser is moved, and the captured cell is put in the microcapillary (12) through the cell introducing hole (44) formed on the microcapillary (12). As mentioned, it is possible to put the selected cells in the microcapillaries (12) one by one. Since the microcapillary (12) is made of a light-transmissive material, the present invention is preferable for cell operation using an optical device as above.

As mentioned above, in a non-contact manner, multiple cells can be selectively taken out at the same time. In particular, this method is preferably applicable to cell inspection in the autologous cell transplant of regeneration medicine and so on.

The present invention is preferably applied to the microchips for handling microobjects such as microchemical chips, iontophoretic chips, immune assay chips, or cellular chips, and is preferably applied to the manufacturing method of these microchips.

What is claimed is:

1. A microchip comprising:
a lower substrate configured as the lower portion of the microchip;
an intermediate section formed on the top of the lower substrate; and
an upper substrate formed on the top of the intermediate section;
wherein the lower substrate, the intermediate section, and the upper substrate are made of light-transmissive and cured resin, and are integrally formed.

2. The microchip according to claim 1, wherein a cavity is formed in the intermediate section, a microstructure is protruded from the wall face of the cavity, and the microstructure is integrated with the wall face.

3. The microchip according to claim 1, wherein the lower substrate is formed in multiple rectangle-shaped blocks divided by grooves formed in a lattice pattern;
wherein the intermediate section is formed in a thin plate having multiple apertures connecting with the grooves dividing the rectangle-shaped blocks;
wherein the upper substrate has a honeycomb structure formed by connecting thin plate walls each other;
wherein the internal space of the honeycomb structure connects with the apertures.

4. The microchip according to claim 1, wherein the lower substrate is formed in a plurality of aligned rectangle blocks divided by the grooves formed in a lattice pattern;
wherein the intermediate section includes the multiple U-shaped thin walls on a plan view connected each other;
wherein the upper substrate is formed to seal the top of the thin wall;
wherein notches are formed on the top end of the thin wall.

5. The microchip according to claim 1, wherein the intermediate section in a trapezoidal cone shape comprises multiple hollow bars;
wherein the upper substrate comprises multiple hollow bars that are extending upward from each of the multiple bars configured as the intermediate section;
wherein the hollow bars configured as the intermediate section and the hollow bars configured as the upper substrate form a microcapillary;
wherein an aperture that a cell is passable is formed on the periphery of the top end of the microcapillary, wherein at least one aperture is formed on the lower portion of the microcapillary than the cell passable aperture.

6. A microchip, wherein the lower substrate includes multiple apertures arrayed in matrix thereon and grooves arrayed in lattice pattern thereon;
wherein the intermediate section includes multiple globular cavity apertures, wherein the upper substrate includes multiple apertures arrayed in matrix thereon and the grooves arrayed in lattice pattern thereon;
wherein the apertures of the lower substrate, the cavity apertures of the intermediate section, and the cavity apertures of the upper substrate are communicated.

7. The microchip according to claim 6, wherein laminated layer comprising the intermediate section with the cavity apertures and the upper substrate with the apertures and the grooves is further piled on the top of the upper substrate.

8. A method of manufacturing microchip comprising:
a formation process of the lower substrate with a certain thickness by curing the light-curing resin;
a formation process of the intermediate section on the top face of the lower substrate in an integrated manner with the lower substrate; and
a formation process of the upper substrate with a certain thickness on the top of the intermediate section in an integrated manner with the intermediate section by curing the light-curing resin;
wherein during the forming process of the intermediate section, integrally laminating an additional cured resin layer on the other cured resin layer repeatedly by proceeding the following steps (a) to (c):
(a) dripping light-curing resin fluid onto the cured resin layer formed in the light-cured resin;
(b) controlling an interval between the top of the cured resin layer and the bottom end of the fluid thickness control plate set above a stage where the lower substrate is set on, making a horizontal relative movement between the stage and the fluid thickness control plate, and attaching the resin fluid on the cured resin layer and the bottom end to control the even thickness of the fluid layer formed on the cured resin layer; and
(c) irradiating a light to the fluid layer to cure the light-cured resin;
wherein non-light-irradiation area and light-irradiation area are set in the step of the integral lamination during the formation process of the intermediate process to form three-dimensional space of the non-irradiation area,
wherein a light is at least partially irradiated to three dimensional space to form a cavity and a microstructure protruded from a wall face of the cavity.

9. The method of manufacturing microchip according to claim 8, wherein the non-light-irradiation area is set in the step of laminating layer in the formation process of the upper substrate to form three-dimensional space of the non-irradiation area;
wherein the three-dimensional space formed in the upper substrate connects the three-dimensional space formed in the intermediate section with the outer surface of the upper substrate.

10. The method of manufacturing microchip according to claim 8, wherein the non-light-irradiation area is set in the laminating layer in the formation process of the lower substrate to form three-dimensional space of the non-irradiation area;
wherein the three-dimensional space formed in the lower substrate connects the three-dimensional space formed in the intermediate section with the outer surface of the lower substrate.
